(19) United States
(12) Patent Application Publication
(10) Pub. No.: US 2007/0073159 A1
(43) Pub. Date: Mar. 29, 2007

(54) Apparatus for recording a tissue containing a fluorescent dye

(76) Inventors: Thomas Ehben, Weisendorf (DE); Sebastian Schmidt, Erlangen (DE)

Correspondence Address: HARNESS, DICKEY & PIERCE, P.L.C., P.O.BOX 8910, RESTON, VA, 20195, US

(21) Appl. No.: 11/525,908

(22) Filed: Sep. 25, 2006

(30) Foreign Application Priority Data
Sep. 26, 2005 (DE) 10 2005 045 906.4

(51) Int. Cl. A61B 5/00

(52) U.S. Cl. 600/473

(57) ABSTRACT

An apparatus is disclosed for recording a tissue containing a fluorescent dye at least in sections. The apparatus includes an illumination device for illuminating the tissue; a first light, suitable for exciting the fluorescent dye; and an image acquisition device. To generate first light with a particularly high light intensity, the illumination device and the image acquisition device are accommodated in separate components.

### PRIORITY STATEMENT

The present application hereby claims priority under 35 U.S.C. §119 on German patent application number DE 10 2005 045 906.4 filed Sep. 26, 2005, the entire contents of which is hereby incorporated herein by reference.

1. Field

The invention generally relates to an apparatus for recording a tissue containing a fluorescent dye at least in sections.

2. Background

During the surgical removal of a tumor, the problem arises that healthy tissue frequently cannot be distinguished with the naked eye from a tissue affected by a tumor. In order to provide a remedy here, the patient is administered before the operation with a fluorescent dye that is specifically enriched in the tumor. During the operation, the exposed tissue is illuminated with a light in the near infrared region that is suitable for exciting the fluorescent dye. The tissue is recorded with the aid of an image acquisition device that has an optical unit for separating a fluorescent image generated by the fluorescent light, and a native tissue image formed by the ambient light. The recorded fluorescent images and the tissue images are superimposed by means of an image processing device, the tumorous tissue being labeled in the superimposed image by means, for example, of a false color display.

The fluorescent light emitted by the fluorescent dye has a substantially lower intensity by comparison with the ambient light. The fluorescent image must be highly intensified for the purpose of generating images. The problem arises here that the ambient light also includes spectral fractions that correspond to the fluorescent light emitted by the fluorescent dye. This interfering fluorescent light is also intensified during the generation of images and falsifies the fluorescent images.

In order to counteract this disadvantage, an attempt is made according to the known systems/methods to raise the intensity of the fluorescent light emitted by the fluorescent dye. Use is made to this end of a strong exciting light source that includes LEDs for generating light in the near infrared region. The LEDs are operated in a pulsed or clocked fashion in order to attain a particularly high power. It is true that success is thereby achieved in distinguishing tissue sections containing fluorescent dye more effectively from other tissue sections. Sections of the tissue that contain only very little fluorescent dye cannot, however, be uniquely distinguished from other tissue sections even with the aid of conventional devices.

### SUMMARY

At least one embodiment of the invention specifies an apparatus, with the aid of which sections of a tissue containing a fluorescent dye, for example, affected by a tumor, can be distinguished from other tissue sections with a sensitivity that is further raised.

According to at least one embodiment of the invention, it is provided that the illumination device forms a first component, and the image acquisition device forms a separate second component. This enables use of an illumination device with a particularly high power and therefore a particularly exact distinction of sections of a tissue, for example of a tumor, that contain a fluorescent dye from other tissue sections.

Because of the separation of the illumination device and the image acquisition device, it is now advantageously possible to design only the image acquisition device as a handheld unit.

A further illumination device for generating a second light can be a constituent of the first component.

The further illumination device can be, for example, a conventional light source for illuminating an operating site.

The image acquisition device can include at least one camera, preferably a CCD camera. Expediently, the image acquisition device has at least two CCD cameras. Furthermore, the image acquisition device can have a first channel for acquiring the first light and a second channel for acquiring the second light. To this end, it is possible to provide, for example, a beam splitter with the aid of which it is possible to separate fluorescent images generated by the first light from native tissue images that can be observed with the second light, for example with the aid of a CCD camera in each case. Here, a first CCD camera can have a sensitivity in the region of a light wavelength of more than 700 nm, in particular, and a second CCD camera can have a sensitivity in the range from 350 to 700 nm, in particular.

According to a further advantageous refinement, the illumination device has LEDs for generating the first light. First light can therefore be generated in a desired spectral region. The first light can, in particular, be light in the infrared region, preferably in the near infrared region. Light with a wavelength of more than 700 nm can be involved in this case.

Furthermore, a clock generator device can be provided for generating a clock frequency given by a periodic sequence of illumination phases and dark phases. The clock frequency can amount to 10 to 100 Hz, preferably 20 to 40 Hz. The proposed clock frequency can no longer be detected by eye, and therefore does not disturb the observation of the illuminated tissue. The clock generator device can be a constituent of the illumination device or of the image acquisition device.

Furthermore, a synchronization device can be provided for generating a synchronization signal corresponding to the clock frequency. The synchronization device can include a transmitter and a receiver for wirelessly transmitting a radio signal forming the synchronization signal. According to a further refinement, the synchronization device can also include an optical acquisition device for acquiring the first light generated at the clock frequency. It is possible thereby to synchronize the illumination device and the image acquisition device. The image acquisition device in this case expediently has a device for separately acquiring first fluorescent images generated during the illumination phases by the excitation of the fluorescent dye, and second fluorescent images generated during the dark phases by illumination of the tissue by the second light. This enables the first fluorescent images recorded during the illumination phases to be corrected by, for example, a subtraction of the second fluorescent images recorded during the illumination phases. It is thereby possible to detect interference signals caused by an excitation with the second light and to eliminate them.

According to an alternative refinement of at least one embodiment, it is also possible that the further illumination device includes a device for generating a further clock frequency given by a periodic sequence of further illumination phases and dark phases, the further clock frequency being phase shifted by 180° by comparison with the clock frequency. In this case, the first light and the second light are thus generated in an alternating fashion. This enables a particularly simple refinement of the image acquisition device. In this case, it can include merely a CCD camera that is sensitive both in the region of visible light and in the region of near infrared light.

### BRIEF DESCRIPTION OF THE DRAWINGS

Example embodiments of the invention are explained below in more detail with reference to the drawings, in which:

FIG. 1 shows a schematic of the essential components of the apparatus, and

FIG. 2 shows a schematic of an image acquisition device.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

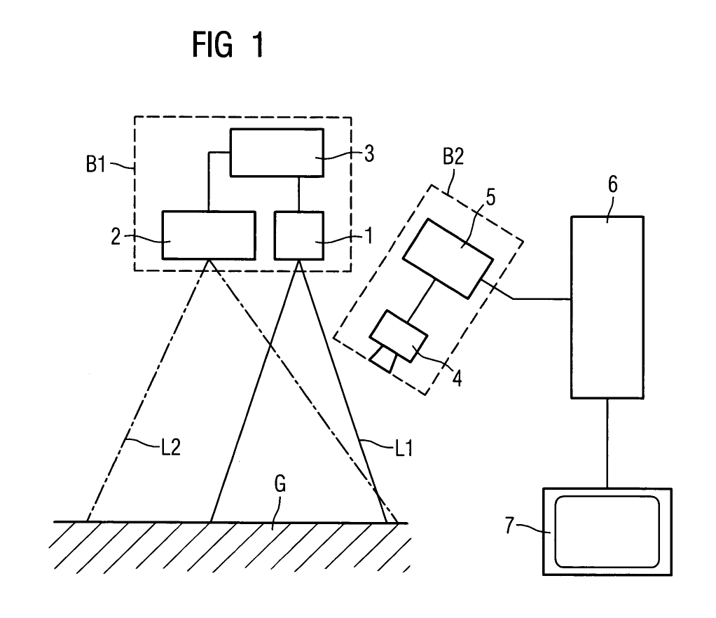

In FIG. 1, a first illumination device 1 includes a multiplicity of high power LEDs (not shown here) for generating a first light L1 in the near infrared region, that is to say in the wavelength region from 700 to 800 nm. A second illumination device 2 can include conventional luminous devices or else LEDs for generating a second light L2. The second light L2 includes wavelengths of visible light, that is to say a wavelength region from 350 to 750 nm, in particular.

A clock generator device 3 is connected to the first illumination device 1 and—in the case of the example embodiment shown here—to the second illumination device 2, as well. The first illumination device 1 and the second illumination device 2 as well as the clock generator device 3 form a first component B1 that can be accommodated in a common housing. This can be, for example, the housing of a lamp for illuminating a patient who is to be operated on.

An image acquisition device 4 having a synchronization device 5 is provided in a second component B2 for the purpose of receiving an image of the section, illuminated with the aid of the first light L1 and the second light L2, of a tissue G. The second component B2 may be designed as a handheld unit. The reference 6 denotes an image processing device that is, for example, a computer with a conventional image processing program. The image processing program can be used, in particular, to superimpose a native tissue image and a fluorescent image reproducing sections of the tissue G that contain the fluorescent dye. An image generated with the aid of an image processing program can be observed on a monitor 7.

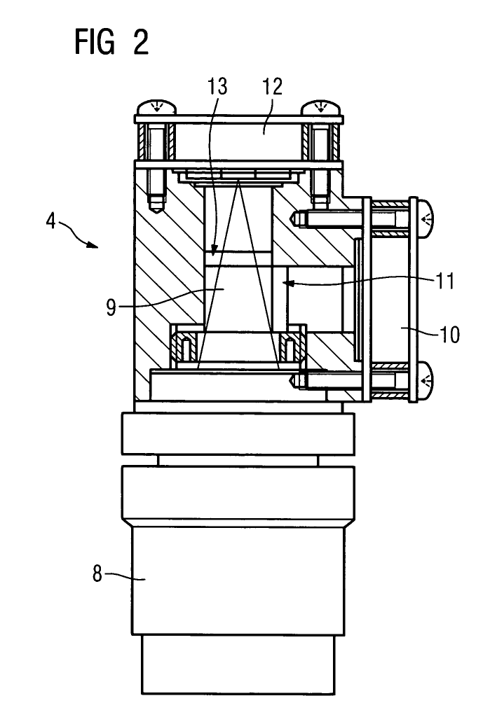

FIG. 2 shows a schematic sectional view of the image acquisition device 4. A beam splitter 9 is arranged downstream of a lens 8 in the beam path. Located between a first camera 10 and the beam splitter 9 is a first filter 11, which is opaque to a wavelength of less than 700 nm. Arranged between a second camera 12 and the beam splitter 9 is a second filter 13, which is opaque in a wavelength region starting from 700 nm.

The functioning of the apparatus is as follows: the clock generator device 3 generates a clock signal, for example at a frequency of 40 Hz, for the periodic switching on and off of the first illumination device 1. If the second illumination device 2 includes LEDs as luminous devices, the clock generator device 3 can also be used to generate a further clock signal with the aid of which the second illumination device 2 is switched on and off relative to the first illumination device 1 in a fashion shifted in phase by 180°.

A fluorescent dye contained in the tissue G is excited with the aid of the first light L1. The first fluorescent image generated thereby is acquired by the first camera 10 of the image acquisition device 4. A signal generated thereby in the first camera 10 can be used to drive the synchronization device 5. The synchronization device 5 in turn drives the image generating device 6 such that the first fluorescent images recorded with the first camera 10 are acquired separately with the aid of the image generating device 6 and processed. In a similar way, it is possible to use the second camera 12 to record native tissue images during the phase-shifted illumination phases generated with the aid of the second illumination device 2, to acquire them separately with the aid of the image generating device 6, and to process them.

Thus, in the case of the example embodiment described native tissue images and first fluorescent images are recorded in an alternating fashion and superimposed by way of the image generating device 6 to form an image.

According to a further refinement, it is also possible for a second light to be generated continuously with the aid of the second illumination device 2. In this case, the clock generator device 3 is not connected to the second illumination device 2.

The second light L2 can contain wavelength components that excite the fluorescent dye contained in the tissue G. The result of this is an interference signal that reduces the informativeness of the first fluorescent images. For the purpose of correction, it is possible during the illumination phases of the first illumination device 1 to use the first camera 10 to record first fluorescent images and, likewise, to record second fluorescent images during the dark phases of the first illumination device 1. The second fluorescent images reproduce the interference signal. The interference signal can be eliminated by subtracting the second fluorescent images from the first fluorescent images. Subsequently, the native tissue images can be superimposed on the corrected first fluorescent images. The synchronization device 5 also serves in this case to acquire the first and second fluorescent images separately by way of the image generating device 6.

In addition to the above-described optical driving of the synchronization device 5, it is also possible to drive it via a radio signal. To this end, the clock generator device 3 can, for example, have an appropriate transmitting apparatus (not shown here), and the synchronization device 5 can have a receiving apparatus (not shown). Instead of the transmitting apparatus and the receiving apparatus, the clock generator device 3 and the synchronization device can, however, also be connected by a cable.

The synchronization device 5 can also be a constituent of the image generating device 4, in particular of the first camera 10 and/or the second camera 12, if the latter are suitable for generating an internal synchronization signal. In particular, CCD cameras operate with an internal clock signal that can be used to generate a synchronization signal. To this end, for example, the first light L1 can be sampled with a high temporal resolution, and the synchronization signal can be generated by frequency multiplication by means of a PLL (phase locked loop).

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An apparatus for recording an image of a tissue containing a fluorescent dye, comprising:
an illumination device to illuminate the tissue;
a first light, suitable to excite the fluorescent dye; and
an image acquisition device to record an image of the tissue, the illumination device forming a first component of the apparatus and the image acquisition device forming a second component, separate from the first component.

2. The apparatus as claimed in claim 1, wherein the image acquisition device is designed as a handheld unit.

3. The apparatus as claimed in claim 1, further comprising:
a further illumination device to generate a second light as a constituent of the first component.

4. The apparatus as claimed in claim 1, wherein the image acquisition device includes at least one camera, preferably a CCD camera.

5. The apparatus as claimed in claim 1, wherein the image acquisition device includes a first channel for acquiring the first light and a second channel for acquiring the second light.

6. The apparatus as claimed in claim 1, wherein the illumination device includes LEDs for generating the first light.

7. The apparatus as claimed in claim 1, wherein the first light is light in the infrared region, preferably in the near infrared region.

8. The apparatus as claimed in claim 1, further comprising:
a clock generator device to generate a clock frequency given by a periodic sequence of illumination phases and dark phases.

9. The apparatus as claimed in claim 8, wherein the clock frequency is 10 to 100 Hz.

10. The apparatus as claimed in claim 8, wherein the clock generator device is a constituent of at least one of the illumination device and the image acquisition device.

11. The apparatus as claimed in claim 8, further comprising a synchronization device to generate a synchronization signal corresponding to the clock frequency.

12. The apparatus as claimed in claim 11, wherein the synchronization device includes a transmitter and a receiver for wirelessly transmitting a radio signal forming the synchronization signal.

13. The apparatus as claimed in claim 11, wherein the synchronization device includes an optical acquisition device for acquiring the first light generated at the clock frequency.

14. The apparatus as claimed in claim 1, wherein the image acquisition device includes at least one device to separately acquire first fluorescent images generated during the illumination phases by the excitation of the fluorescent dye, and second fluorescent images generated during the dark phases by illumination of the tissue by the second light.

15. The apparatus as claimed in claim 1, wherein the further illumination device includes a device to generate a further clock frequency given by a periodic sequence of further illumination phases and dark phases, the further clock frequency being phase shifted by 180° by comparison with the clock frequency.

16. The apparatus as claimed in claim 1, wherein the image acquisition device includes at least one CCD camera.

17. The apparatus as claimed in claim 1, wherein the first light is light in the near infrared region.

18. The apparatus as claimed in claim 8, wherein the clock frequency is 20 to 40 Hz.

19. An apparatus for recording an image of a tissue containing a fluorescent dye, comprising:
illumination means for illuminating the tissue;
means for exciting the fluorescent dye; and
acquisition means for recording an image of the tissue, the illumination means being in a first component of the apparatus and the acquisition means being in a second component, separate from the first component.

20. The apparatus as claimed in claim 19, further comprising:
means for generating a second light as a constituent of the first component.
